(19) United States
(12) Patent Application Publication
(10) Pub. No.: US 2025/0067749 A1
(43) Pub. Date: Feb. 27, 2025

(54) A Diazo Compound And A Preparation Method And A Use Thereof

(71) Applicant: PEKING UNIVERSITY, Beijing (CN)

(72) Inventors: Zhixing CHEN, Beijing (CN); Li Quan, Beijing (CN); Cong Li, Beijing (CN); Kunlun Cheng, Beijing (CN); Qijin Zhao, Beijing (CN)

(21) Appl. No.: 18/722,154

(22) PCT Filed: Apr. 28, 2023
(86) PCT No.: PCT/CN2023/091869

§ 371 Date: Jun. 20, 2024

(30) Foreign Application Priority Data
Apr. 29, 2022 (CN) 202210469035.4

(51) Int. Cl. G01N 33/68; C07C 249/02; C07C 251/24; C07D 215/14; G01N 33/53; G01N 33/531

(52) U.S. Cl. CPC G01N 33/6848; C07C 249/02; C07C 251/24; C07D 215/14; G01N 33/5308; G01N 33/531

(57) ABSTRACT

A diazo compound and a preparation method and a use thereof. The diazo compound has the structure as shown in formula

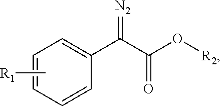

wherein R₁ represents H, alkyl, halogen, alkoxy or alkylamino; and R₂ represents an aromatic group. On the basis of the aforementioned diazo compound as a derivatization reagent, the derivatization treatment of a small molecule carboxylic acid can significantly enhance the mass spectrometry response thereof and improve the detection sensitivity, thereby improving the detection accuracy. Moreover, it is not required to configure special chromatographic columns or special mobile phases for the derivatized small molecule carboxylic acid, and the best separation effect in a shorter time can be achieved on the basis of a lower cost, which is more conducive to high-throughput sample detection and has better detection accuracy.

### CROSS-REFERENCE TO RELATED APPLICATION

This application is a U.S. national phase patent application of PCT/CN2023/091869 filed Apr. 28, 2023 which claims the benefit on the basis of Chinese application with CN Application Number 202210469035.4 and an application date of Apr. 29, 2022, and claims its priority, the disclosure of the CN application is incorporated herein in its entirety again.

### TECHNICAL FIELD

The present disclosure relates to the field of analytical chemistry, and specifically to a diazo compound and a preparation method and a use thereof.

### BACKGROUND

The quantitative detection of small molecule carboxylic acid metabolites mainly includes two technical routes: enzymatic coupling technology and chromatography-tandem mass spectrometry technology. Compared with enzymatic coupling technology, chromatography-tandem mass spectrometry technology has high sensitivity and high throughput, and can simultaneously detect multiple metabolites, greatly reducing sample consumption.

Enzymatic coupling technology utilizes the specific recognition of small molecule carboxylic acid metabolites by enzymes, associates the concentration of small molecule carboxylic acid metabolites with the enzymatic reaction rate, and ultimately indirectly reflects the concentration of small molecule carboxylic acid metabolites through the magnitude of changes in specific wavelength light absorption (i.e. magnitude of the enzymatic reaction rate). Generally speaking, the concentration of small molecule carboxylic acid metabolites in biological samples by quantitative detection using enzymatic coupling technology ranges from 10 μM to 100 μM. The enzymatic coupling technology is characterized by simple operation, relatively few steps, low requirements for experimental conditions and personnels, instrument availability, and completion of the experiment basically on a biological experimental bench.

The chromatography-tandem mass spectrometry technology combines the characteristics of chromatography separation and qualitative and quantitative analysis by mass spectrometry, and is currently recognized as the gold standard instrument for qualitative and quantitative analysis in various industries. Chromatography can separate different substances in a sample, specifically manifested by unique retention time of each substance, and can provide molecular weight information of chromatographic elution components in real time when combined with mass spectrometry, which can be used to assist in determining the chemical information of the elution components. Among them, liquid chromatography is the most widely used chromatography technology in biological and pharmaceutical analysis. Triple quadrupole mass spectrometry and high-resolution mass spectrometry are currently the mainstream mass spectrometry technologies, wherein the former has high sensitivity and advantages in the quantification of known compounds, while the latter has high resolution and advantages in the qualitative analysis of unknown compounds.

The chromatography-tandem mass spectrometry technology is a commonly used technique for quantitative detection of small molecule metabolites. Common liquid chromatography methods for small molecule carboxylic acid metabolites include reverse phase chromatography, hydrophilic interaction chromatography, and ion-pair chromatography. Due to the strong hydrophilicity and similar polarity of small molecule carboxylic acid metabolites, they cannot be effectively distinguished by reverse phase chromatograph, resulting in the co-elution and mutual inhibition of ionization of different small molecule carboxylic acid metabolites. Although hydrophilic interaction chromatography and ion-pair chromatography both can separate different small molecule carboxylic acid metabolites, they require special mobile phases and chromatographic columns. Hydrophilic interaction chromatography requires special hydrophilic interaction chromatographic columns and alkaline mobile phases, while ion-pair chromatography requires the addition of ion pairing reagents such as n-butylamine, etc., to the mobile phase, which limits its use in combination with mass spectrometry technology and has been largely phased out of the range of liquid chromatography-tandem mass spectrometry. Currently, there are two main ideas for detecting characteristic metabolite small molecule carboxylic acids based on liquid chromatography-tandem mass spectrometry technology:

Idea 1, a classical method of extracting the sample and then directly injecting same. The advantage of this method lies in that the sample pretreatment is simple and fast, generally includes mixing the sample with an organic solvent at 1:3 and then subjecting same to vortex centrifugation, the core purpose of which is to extract the desired target compound and remove the solid impurities such as cell membranes and proteins, etc. One disadvantage of this method lies in that under the negative ion mode of mass spectrometry, the detection sensitivity is not high, only about 10 times higher than that of enzyme-linked immunosorbent assay, with a detection limit of 100 nM to 10 μM. This is the inherent limitation in the sensitivity of the negative ion mode of mass spectrometry itself, and cannot be further improved through optimization methods. Another disadvantage of this method lies in that in order to improve retention, HILIC hydrophilic chromatography mode will be used in the direct detection method, which has a detection time of at least 20 minutes or more, the time consumption of which is increased by 2-3 times compared to commonly used reverse phase chromatography, which is not conducive to high-throughput testing.

Idea 2, derivatization pre-treatment of the sample and then the mass spectrometry analysis. The derivatization methods that belong to this idea include oBHA (“Derivatization of the tricarboxylic acid intermediates with O-benzylhydroxylamine for liquid chromatography-tandem mass spectrometry detection”, Analytical Biochemistry, 2014), etc. However, the oBHA derivatization method has the following disadvantages: the detection limit of lactic acid is relatively high, which is not suitable for trace analysis and cannot be used for in situ derivatization of 96-well cultured cells; the derivatization process of biological samples requires multiple extractions, spin drying, and re-dissolution, which takes a long operation time and can easily lead to the degradation of metabolites.

In summary, there are problems of low mass spectrometry response and sensitivity, long instrumental analysis time that is not conducive to high-throughput sample detection, increased analysis costs due to the need for special chromatography columns, potential degradation of unstable small molecule carboxylic acid metabolites during sample treatment due to the long treatment time of biological samples and increased analysis costs due to the need for configuration of special mobile phases, etc., in the prior art. Therefore, it is necessary to provide a new derivatization reagent to improve the above problems.

### SUMMARY

The main object of the present disclosure is to provide a diazo compound and a preparation method and a use thereof, in order to solve the problems of low mass spectrometry response and sensitivity, long instrumental analysis time that is not conducive to high-throughput sample detection, increased analysis costs due to the need for special chromatography columns, potential degradation of unstable small molecule carboxylic acid metabolites during sample treatment due to the long treatment time of biological samples and increased analysis costs due to the need for configuration of special mobile phases, etc., when small molecule carboxylic acids, especially small molecule carboxylic acid metabolites are detected in the prior art.

In order to achieve the above object, according to one aspect of the present disclosure, a diazo compound is provided, the diazo compound has the structure as shown in formula I, wherein in formula I, R₁ represents H, alkyl, halogen, alkoxy or alkylamino; and R₂ represents an aromatic group.

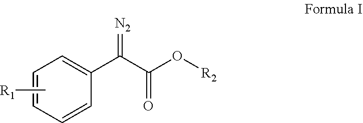

Further, R₁ represents H, C₁-C₆ alkyl, halogen, C₁-C₆ alkoxy or dimethylamino; and R₂ represents methylenequinolyl or ethyl-N,N-dimethylanilino group.

In order to achieve the above object, according to one aspect of the present disclosure, a preparation method of the above diazo compound is provided, which includes: performing an esterification reaction on the first dispersion containing a phenylacetic acid compound and an alcohol compound to generate an intermediate product A; performing a diazotization reaction on the second dispersion containing the intermediate product A and the diazo transfer reagent to generate a diazo compound; wherein, the phenylacetic acid compound has the structure as shown in

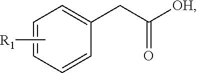

and the alcohol compound has the structure as shown in R₂—OH; R₁ represents H, alkyl, halogen, alkoxy or alkylamino; and R₂ represents an aromatic group.

Further, during the esterification reaction process, the reaction temperature is from 0° C. to 30° C. and the reaction time is from 0.5 hours to 24 hours; preferably, during the diazotization reaction process, the reaction temperature is from 0° C. to 30° C. and the reaction time is from 1 hour to 24 hours; preferably, during the esterification reaction process, the molar ratio of the phenylacetic acid compound to the alcohol compound is (0.5-2): 1; preferably, during the diazotization reaction process, the molar ratio of the intermediate product A to the diazo transfer reagent is (1-3): 1; preferably, during the diazotization reaction process, the diazo transfer reagent is one or more of 4-acetamidobenzenesulfonyl azide, p-toluenesulfonyl azide, 4-carboxybenzenesulfonyl azide, 1H-imidazole-1-sulfonyl azide hydrochloride, or 2-azido-1,3-dimethylimidazolium hexafluorophosphate; preferably, the first dispersion comprises a first solvent, which is one or more of dichloromethane, trichloromethane, N, N-dimethylformamide, tetrahydrofuran, or diethyl ether; preferably, the first dispersion also comprises a first catalyst, which is one or more of triethylamine, N,N-diisopropylethylamine, or alkali carbonate; preferably, the second dispersion comprises a second solvent, which is acetonitrile and/or dimethyl sulfoxide; and preferably, the second dispersion also comprises a second catalyst, which is further preferably one or more of 1,8-diazabicyclo[5.4.0]undec-7-ene, triethylamine, sodium bicarbonate, sodium carbonate, potassium carbonate, potassium hydroxide or potassium acetate.

According to another aspect of the present disclosure, a detection method for quantitative analysis of a small molecule carboxylic acid is provided, wherein the small molecule carboxylic acid represents a carboxylic acid with a molecular weight between 46 and 500, and the detection method includes: derivatization treatment involving derivatizing a sample containing the small molecule carboxylic acid with a derivatization reagent to obtain a derivatized sample; liquid chromatography-mass spectrometry analysis involving performing liquid chromatography-mass spectrometry analysis on the derivatized sample to obtain a liquid chromatogram-mass chromatogram, and quantitatively analyzing the small molecule carboxylic acid components in the sample based on the liquid chromatogram-mass chromatogram; wherein, the derivatization reagent is the aforementioned diazo compound, or the diazo compound prepared by the aforementioned preparation method.

Further, derivatization treatment includes formulating 20-100 mM of an acetonitrile solution of the derivatization reagent and recording it as solution A; formulating 20-100 mM of an aqueous solution of the hydroxylamine compound and recording it as solution B; mixing the sample containing the small molecule carboxylic acid with the solution A, centrifuging the mixture at 10,000 to 17,000 rpm and a temperature of 4° C. to 30° C. for 5 to 8 minutes, then mixing the supernatant after centrifugation with the solution B, and subjecting the sample to derivatization reaction at a temperature of 50° C. to 80° C. to obtain the derivatized sample; preferably the time for the derivatization reaction being 10 minutes to 60 minutes; preferably, the sample being plasma, serum, urine, tears, tissue fluid, cells, tissue homogenate, bacterial culture medium, blood plaque or feces; and preferably, the small molecule carboxylic acid being one or more of myristic acid, palmitic acid, palmitoleic acid, stearic acid, oleic acid, linoleic acid, arachidonic acid, lactic acid, pyruvic acid, fumaric acid, oxaloacetic acid, α-ketoglutaric acid, succinic acid, malic acid, citric acid, or isocitric acid.

Further, during the liquid chromatography-mass spectrometry analysis process, the mobile phase used for the liquid chromatography analysis comprises phase A and phase B, wherein, the phase A is a mixed solution of water and formic acid, the phase B is a mixed solution of acetonitrile and formic acid, and the liquid chromatography elution program used in the liquid chromatography analysis process is a gradient elution program, the liquid chromatography elution program comprises a first equilibrium process, a first elution process, a second elution process, and a second equilibrium process sequentially; the volume of phase A is recorded as V_{A} and the volume of phase B is recorded as V_{B}, and the flow rate of the liquid chromatography mobile phase is recorded as Vₙ, with V₁ ranging from 0.2 to 0.6 mL/min; preferably, in a mixed solution of water and formic acid, the volume ratio of water to formic acid is 200: (0.1-0.3); and preferably, in a mixed solution of acetonitrile and formic acid, the volume ratio of acetonitrile to formic acid is 200: (0.1-0.3).

Further, during the first equilibrium process, V_{A} is 70%-80%, V_{B} is 20%-30%, and the time of the first equilibrium process is 0-1 minute; during the first elution process, V_{A} is in a dynamic change process gradually switching from 70%-80% to 20%-30%, while V_{B} is in a dynamic change process gradually switching from 20%-30% to 70%-80%, and the time of the first elution process is 2-5 minutes; during the second elution process, V_{A} is in a dynamic change process gradually switching from 20%-30% to 0-5%, while V_{B} is in a dynamic change process gradually switching from 70%-80% to 95%-100%, and the time of the second elution process is 1-3 minutes; during the second equilibrium process, V_{A} is in a dynamic change process gradually switching from 0-5% to 70%-80%, while V_{B} is in a dynamic change process gradually switching from 95%-100% to 20%-30%, and the time of the second equilibrium process is 1-1.5 minutes; or during the first equilibrium process, V_{A} is 45%-55%, V_{B} is 45%-55%, and the time of the first equilibrium process is 0-1 minute; during the first elution process, V_{A} is in a dynamic change process gradually switching from 45%-55% to 0-5%, while V_{B} is in a dynamic change process gradually switching from 45%-55% to 95%-100%, and the time of the first elution process is 3-4 minutes; during the second elution process, V_{A} is 0-5%, while V_{B} is 95%-100%, and the time of the second elution process is 2-4 minutes; during the second equilibrium process, V_{A} is in a dynamic change process gradually switching from 0-5% to 50%-55%, while V_{B} is in a dynamic change process gradually switching from 95%-100% to 45%-55%, and the time of the second equilibrium process is 1-1.5 minutes.

According to another aspect of the present disclosure, use of the aforementioned diazo compound, or the diazo compound prepared by the aforementioned preparation method in drug screening related to the function of mitochondrial respiratory chain complexes is provided.

Further, the use includes in situ detection of metabolites of living cells by utilizing the diazo compound; preferably, the drug comprises an agonist and/or an inhibitor; preferably, the metabolites are carboxylic acid metabolites in the tricarboxylic acid cycle; preferably, the carboxylic acid metabolites in the tricarboxylic acid cycle refer to a small molecule carboxylic acid with a molecular weight between 46 and 500; more preferably, the small molecule carboxylic acid is one or more of myristic acid, palmitic acid, palmitoleic acid, stearic acid, oleic acid, linoleic acid, arachidonic acid, lactic acid, pyruvic acid, fumaric acid, oxaloacetic acid, α-ketoglutaric acid, succinic acid, malic acid, citric acid, or isocitric acid; and more preferably, the above detection method for quantitative analysis of a small molecule carboxylic acid is used for in situ detection.

According to another aspect of the present disclosure, a kit is provided, which comprises the aforementioned diazo compound, or the diazo compound prepared by the aforementioned preparation method.

On the basis of the aforementioned diazo compound as a derivatization reagent, the in situ derivatization of cell samples can be more effectively achieved in the present disclosure, thereby shortening the treatment process of biological samples, reducing the degradation of small molecule carboxylic acids (such as small molecule carboxylic acid metabolites at any time point in the vital or metabolic activities of organisms in vivo) during the sample treatment process, and further improving the detection accuracy.

### BRIEF DESCRIPTION OF THE DRAWINGS

The accompanying drawings of the description, which form a part of the application, are used to provide a further understanding of the disclosure. The illustrative embodiments and their descriptions of the disclosure are used to explain the disclosure, and do not constitute an improper limitation thereto. In the accompanying drawings:

FIG. 1 shows the flowchart of a 96-well plate live cell metabolic flow assay experiment using U—C13-glucose as the carbon source according to the present disclosure;

FIG. 2 shows the schematic diagram of the principle of a 96-well plate live cell metabolic flow assay experiment using U—C13-glucose as the carbon source according to the present disclosure;

FIG. 3 shows the schematic diagram of the metabolic flow of tricarboxylic acid cycle substrate in neonatal rat cardiomyocytes treated simultaneously with the inhibitor of mitochondrial respiratory chain complex I (rotenone) and the inhibitor of complex III (antimycin A) according to the present disclosure;

FIG. 4 shows the schematic diagram of the metabolic flow of tricarboxylic acid cycle substrate in neonatal rat cardiomyocytes treated with the inhibitor of mitochondrial respiratory chain complex V (oligomycin) according to the present disclosure;

FIG. 5 shows the schematic diagram of the metabolic flow of tricarboxylic acid cycle substrate in neonatal rat cardiomyocytes treated with the uncoupling agent (carbonylcyanide-4-trifluoromethoxyphenylhydrazone (FCCP)) according to the present disclosure;

FIG. 6 shows the schematic diagram of the absolute content of tricarboxylic acid cycle metabolites in normal human serum measured in an example of the present disclosure;

FIG. 7 shows an LC/MS schematic diagram of the diazo compound in example 1 of the present disclosure;

FIG. 8 shows an LC/MS schematic diagram of the diazo compound in example 2 of the present disclosure;

FIG. 9 shows the secondary mass spectrogram-chromatogram of the pyruvic acid standard sample (1 nM) derivatized with the diazo compound in example 1 of the present disclosure;

FIG. 10 shows the secondary mass spectrogram-chromatogram of the α-ketoglutaric acid standard sample (1 nM) derivatized with the diazo compound in example 1 of the present disclosure;

FIG. 11 shows the secondary mass spectrogram-chromatogram of the α-ketovaline standard sample (2 nM) derivatized with the diazo compound in example 1 of the present disclosure;

FIG. 12 shows the secondary mass spectrogram-chromatogram of the succinic acid standard sample (5 nM) derivatized with the diazo compound in example 1 of the present disclosure; and

FIG. 13 shows the secondary mass spectrogram-chromatogram of the lactic acid standard sample (5 nM) derivatized with the diazo compound in example 1 of the present disclosure.

### DETAILED DESCRIPTION OF THE EMBODIMENTS

It should be noted that the examples and features in the examples in the application can be combined with each other without conflicting. The disclosure will be described in detail below with reference to the drawings and in combination with examples.

As described in the background section of the present disclosure, there are problems of low mass spectrometry response and sensitivity, long instrumental analysis time that is not conducive to high-throughput sample detection, increased analysis costs due to the need for special chromatography columns, potential degradation of unstable small molecule carboxylic acid metabolites during sample treatment due to the long treatment time of biological samples and increased analysis costs due to the need for configuration of special mobile phases, etc., when small molecule carboxylic acids, especially small molecule carboxylic acid metabolites are detected in the prior art. To solve such problems, the present disclosure provides a diazo compound, which has the structure as shown in formula I, wherein R₁ represents H, alkyl, halogen, alkoxy or alkylamino; and R₂ represents an aromatic group.

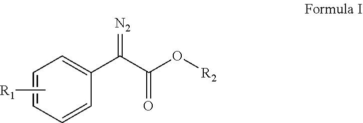

When liquid chromatography-tandem mass spectrometry technology is used for quantitative analysis of small molecule carboxylic acids, the inventors of the present disclosure creatively discovered that on the basis of the aforementioned diazo compound as a derivatization reagent, derivatization treatment was carried out on the test sample containing small molecule carboxylic acids (derivatization treatment being the esterification reaction between the diazo compound and the small molecule carboxylic acids in the test sample containing the small molecule carboxylic acids) to obtain a derivatized sample. As such, during the subsequent detection process, the mass spectrometry response of small molecule carboxylic acids in the derivatized sample can be significantly enhanced, and the detection sensitivity can be improved, thereby improving the detection accuracy. Moreover, it is not required to configure special chromatographic columns and special mobile phases for the derivatized small molecule carboxylic acid, and the best separation effect in a shorter time can be achieved on the basis of a lower cost, which is more conducive to high-throughput sample detection.

Especially, on the basis of the aforementioned diazo compound as a derivatization reagent, the in situ derivatization of cell samples can be more effectively achieved in the present disclosure, thereby shortening the treatment process of biological samples, reducing the degradation of small molecule carboxylic acids (such as small molecule carboxylic acid metabolites at any time point in the vital or metabolic activities of organisms in vivo) during the sample treatment process, and further improving the detection accuracy.

In some preferred embodiments, R₁ represents H, C₁-C₆ alkyl, halogen, C₁-C₆ alkoxy or dimethylamino; and R₂ represents methylenequinolyl or ethyl-N,N-dimethylanilino group. On this basis, the derivatized small molecule carboxylic acids have higher mass spectrometry response, higher detection sensitivity, and better detection accuracy.

In some alternative embodiments, the diazo compounds may be selected from the following compounds:

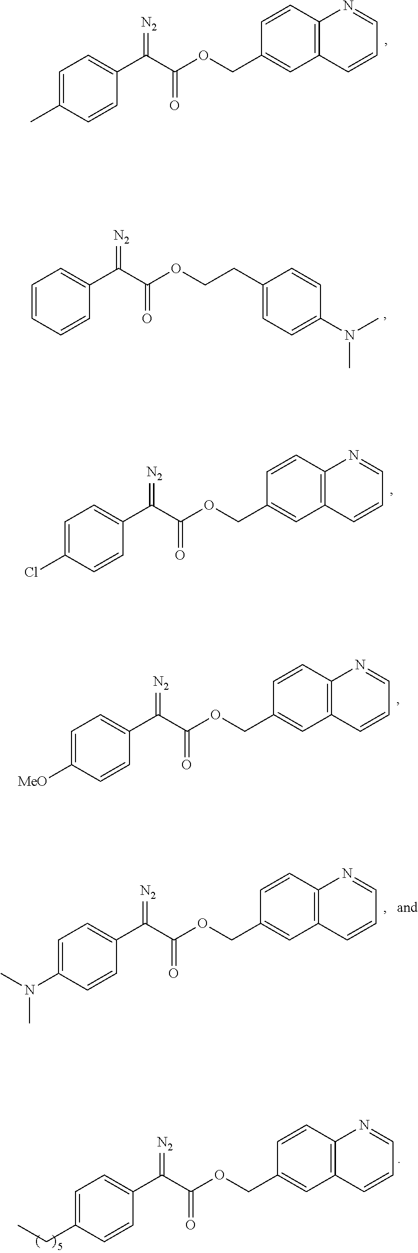

The present disclosure also provides a preparation method of the above diazo compound, which includes: performing an esterification reaction on the first dispersion containing a phenylacetic acid compound and an alcohol compound to generate an intermediate product A; performing a diazotization reaction on the second dispersion containing the intermediate product A and the diazo transfer reagent to generate a diazo compound; wherein, the phenylacetic acid compound has the structure as shown in

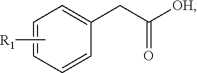

and the alcohol compound has the structure as shown in R₂—OH; R₁ represents H, alkyl, halogen, alkoxy or alkylamino; and R₂ represents an aromatic group. Intermediate product A has the structure as shown in formula

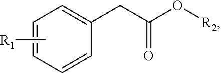

and the synthesis route is as follows:

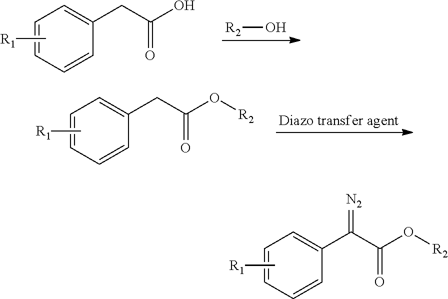

In view of the above reasons, when the characteristic metabolite small molecule carboxylic acid is detected through liquid chromatography-tandem mass spectrometry technology with the diazo compound obtained by the aforementioned preparation method in the present disclosure as a derivatization reagent, the derivatization treatment of the test sample containing small molecule carboxylic acids can significantly enhance the mass spectrometry response thereof and improve the detection sensitivity, thereby improving the detection accuracy. Moreover, it is not required to configure special chromatographic columns or special mobile phases for the derivatized small molecule carboxylic acid, and the best separation effect in a shorter time can be achieved on the basis of a lower cost, which is more conducive to high-throughput sample detection and has better detection accuracy. Especially, on the basis of the aforementioned diazo compound as a derivatization reagent, the beneficial effect of in situ derivatization of cell samples can be effectively achieved, thereby shortening the treatment process of biological samples, reducing the degradation of small molecule carboxylic acids in the biological sample during the sample treatment process, and further improving the detection accuracy. In addition, the preparation method has a simpler operation process, easier access to raw materials, and higher yield and purity of the obtained product (diazo compound).

In an alternative embodiment, those skilled in the art can directly subject the phenylacetic acid compounds and the alcohol compounds to esterification reaction so as to generate the aforementioned intermediate product A. Those skilled in the art can also firstly prepare the phenylacetic acid compounds into acyl chloride compounds, and then subject the acyl chloride compounds and the alcohol compounds to esterification reaction so as to generate the aforementioned intermediate product A. The synthesis route is as follows:

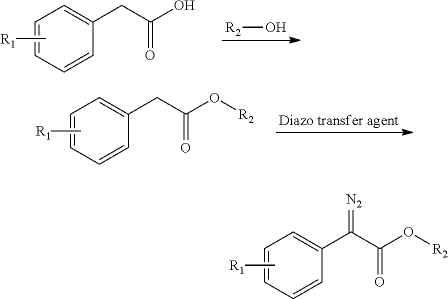

In order to further improve the reaction stability of esterification and diazotization reactions and thus further increase the yield of products, in a preferred embodiment, the reaction temperature during the esterification reaction is 0-30° C., which can be 0° C., 5° C., 10° C., 15° C., 20° C., 25° C., or 30° C., for example; and the reaction time is 0.5-24 hours, which can be 0.5 hours, 5 hours, 10 hours, 15 hours, 20 hours, or 24 hours, for example. During the diazotization reaction, the reaction temperature is 0-30° C., which can be 0° C., 5° C., 10° C., 15° C., 20° C., 25° C., or 30° C., for example; and the reaction time is 1-24 hours, more preferably the reaction time is 10-24 hours, which can be 10 hours, 15 hours, 20 hours, or 24 hours, for example.

In order to further improve the product yield of esterification and diazotization reactions, in a preferred embodiment, the molar ratio of phenylacetic acid compounds to alcohol compounds during the esterification reaction is (0.5-2): 1, which can be 0.5:1, 1:1, 1.5:1 or 2:1, for example. During the diazotization reaction, the molar ratio of intermediate product A to diazo transfer reagent is (1-3): 1, which can be 1:1, 2:1, or 3:1, for example.

In order to further efficiently improve the product yield and purity of esterification and diazotization reactions, in a preferred embodiment, during the diazotization reaction process, the diazo transfer reagent is one or more of 4-acetamidobenzenesulfonyl azide, p-toluenesulfonyl azide, 4-carboxybenzenesulfonyl azide, 1H-imidazole-1-sulfonyl azide hydrochloride, or 2-azido-1,3-dimethylimidazolium hexafluorophosphate. In a preferred embodiment, the first dispersion comprises a first solvent, which is one or more of dichloromethane, trichloromethane, N, N-dimethylformamide, tetrahydrofuran, or diethyl ether. In a preferred embodiment, the first dispersion also comprises a first catalyst, which is one or more of triethylamine, N,N-diisopropylethylamine, or alkali carbonate such as potassium carbonate, sodium carbonate, and cesium carbonate, etc. In a preferred embodiment, the second dispersion comprises a second solvent, which is acetonitrile and/or dimethyl sulfoxide. In a preferred embodiment, the second dispersion also comprises a second catalyst, which is further preferably one or more of 1,8-diazabicyclo[5.4.0]undec-7-ene, triethylamine, sodium bicarbonate, sodium carbonate, potassium carbonate, potassium hydroxide or potassium acetate.

Some synthesis routes of the aforementioned diazo compounds can be listed here, for example, in some embodiments of the present disclosure, the synthesis route of diazo compound (DQclB) is as follows:

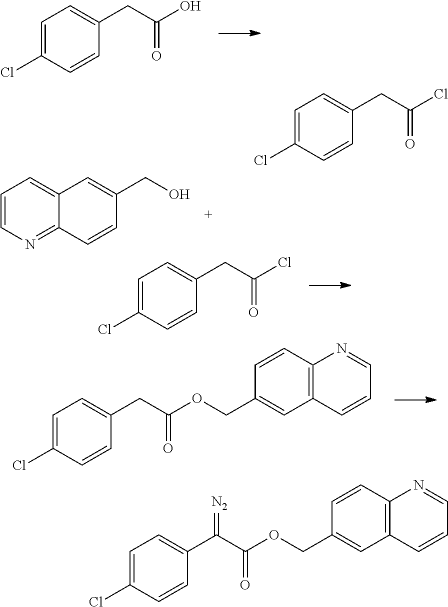

The synthesis route of diazo compound (DQmoB) is as follows:

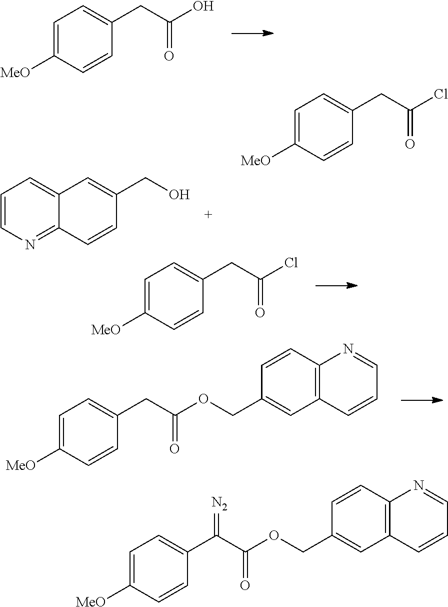

The synthesis route of diazo compound (DQdmaB) is as follows:

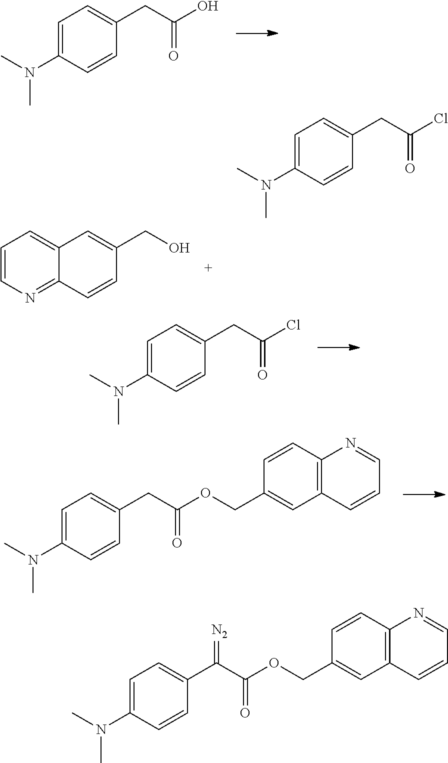

The synthesis route of diazo compound (DQhB) is as follows:

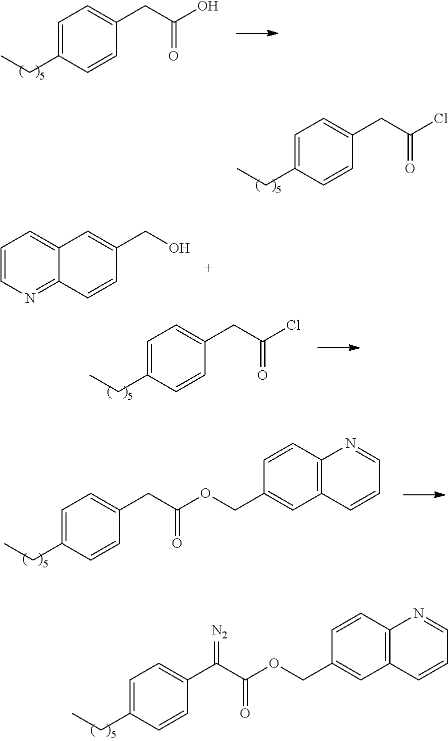

Specifically, in an alternative embodiment of the present disclosure, by way of example, phenylacetic acid is used as a raw material of the reaction, the phenylacetic acid (1 equivalent) can be dissolved in dichloromethane, and oxalyl chloride (1.1 equivalents) and N, N-dimethylformamide (DMF) (0.01 equivalents) can be added dropwise at 0-5° C. The reaction mixture is stirred at 20° C. for 12 hours, and the crude product of the reaction is rotary evaporated under reduced pressure to obtain an acyl chloride compound. Then, quinolinol (1 equivalent) is dissolved in dichloromethane, triethylamine (2.5 equivalents) is added, and the aforementioned acyl chloride compound (1.1 equivalents) is slowly added at low temperature. The reaction mixture is stirred at 4-30° C. for 8-24 hours. The reaction solution is poured into pure water, the aqueous phase is separated, which is extracted twice sequentially with dichloromethane. The organic phases are combined, dried over anhydrous sodium sulfate, filtered, and rotary evaporated to dryness under reduced pressure. The dried substance obtained from rotary evaporation is purified by column chromatography to obtain quinoline p-chlorophenylacetate.

The aforementioned quinoline p-chlorophenylacetate is dissolved in acetonitrile and 4-acetamidobenzenesulfonyl azide (p-ABSA) (2 equivalents) and 1,8-diazabicyclo[5.4.0]undec-7-ene (DBU) (3 equivalents) are added sequentially at low temperature. The reaction mixture is stirred at 4-30° C. for 8-24 hours. The reaction solution is concentrated under reduced pressure, and rotary evaporated to dryness, the crude product is purified by column chromatography to obtain the aforementioned diazo compound. In practical applications, those skilled in the art can use raw materials with different functional groups (phenylacetic acid compounds and alcohol compounds), and by using the aforementioned preparation method, corresponding diazo compounds can be obtained, which will not be further elaborated here.

The present disclosure also provides a detection method for quantitative analysis of a small molecule carboxylic acid, wherein the small molecule carboxylic acid represents a carboxylic acid with a molecular weight between 46 and 500. This detection method includes derivatization treatment and liquid chromatography-mass spectrometry analysis. Derivatization treatment includes using a derivatization reagent to derivatize the sample and obtain the derivatized sample. The liquid chromatography-mass spectrometry analysis includes performing liquid chromatography-mass spectrometry analysis on the derivatized sample to obtain a liquid chromatogram-mass chromatogram, and quantitatively analyzing the small molecule carboxylic acid components in the sample based on the liquid chromatogram-mass chromatogram; wherein, the derivatization reagent is the aforementioned diazo compound, or the diazo compound prepared by the aforementioned preparation method.

In view of the above reasons, when the characteristic metabolite small molecule carboxylic acid is detected through liquid chromatography-tandem mass spectrometry technology, the inventors of the present disclosure creatively discovered that on the basis of the aforementioned diazo compound as a derivatization reagent, the derivatization treatment of the test sample containing small molecule carboxylic acids can significantly enhance the mass spectrometry response thereof after the derivatization treatment and improve the detection sensitivity, thereby improving the detection accuracy. Moreover, it is not required to configure special chromatographic columns and special mobile phases for the derivatized small molecule carboxylic acid, and the best separation effect in a shorter time can be achieved on the basis of a lower cost, which is more conducive to high-throughput sample detection. The sample treatment is simple and fast, and is suitable for detection by various types of liquid chromatograph-mass spectrometer, and can detect various small molecule carboxylic acids in a short time (less than 30 minutes) with high throughput.

Specifically, the above derivatized small molecule carboxylic acid is subjected to liquid chromatography-mass spectrometry analysis to obtain a liquid chromatogram-mass chromatogram, and the peaks in the liquid chromatogram-mass chromatogram are integrated with commercial softwares, and the small molecule carboxylic acids in the sample are quantitatively analyzed based on the integral area, which can be achieved by those skilled in the art according to well-known knowledge, and will not be further elaborated here.

In a preferred embodiment, the derivatization treatment includes formulating 20-100 mM of an acetonitrile solution of the derivatization reagent and recording it as solution A; formulating 20-100 mM of an aqueous solution of the hydroxylamine compound and recording it as solution B; mixing the sample containing the small molecule carboxylic acid with the solution A, centrifuging the mixture at 10,000 to 17,000 rpm and a temperature of 4° C. to 30° C. for 5 minutes to 8 minutes, then mixing the supernatant after centrifugation with the solution B, and subjecting the sample to derivatization reaction (specifically the esterification reaction) at a temperature of 50° C. to 80° C. to obtain the derivatized sample, preferably the time for the derivatization reaction being 10 minutes to 60 minutes. On this basis, the efficiency of the derivatization reaction is higher, after the aforementioned derivatization treatment of the sample, the mass spectrometry response of the small molecule carboxylic acid metabolites can be further enhanced, and at the same time the detection sensitivity can be further improved, thereby improving the detection accuracy. At the same time, the best separation effect of components in the test sample can be achieved in a shorter time on the basis of lower cost.

In some alternative embodiments, the samples may be various types of biological samples that can be ground and extracted, such as plasma, serum, urine, tears, tissue fluid, cells, tissue homogenate (fragments), bacterial culture medium, blood plaque or feces, etc. Of course, the test object of the aforementioned testing method in the present disclosure can also be non-biological samples, such as environmental samples (e.g. carboxylic acids in water and air samples) and food samples (e.g. carboxylic acids in additives and preservatives).

In order to further improve the stability during the detection process and further enhance the detection accuracy at the same time, in a preferred embodiment, during the liquid chromatography-mass spectrometry analysis process, a triple quadrupole mass spectrometer at a source temperature of 150-160° C., a cone voltage of 30-35 kV, a capillary voltage of 2-5 kV, a desolvation temperature of 400-450° C., a cone gas flow rate of 20-25 L/Hr, and a desolvation gas flow rate of 1,000-1,100 L/Hr can be used in the liquid chromatography-mass spectrometry analysis process. It should be additionally noted that the above conditions are the testing conditions for the Waters Xevo TQ-S micro mass spectrometer, those skilled in the art can adjust and optimize the corresponding parameters based on practical experiences when using other mass spectrometers.

In order to further improve the stability of the detection process and enhance the detection accuracy at the same time, in a preferred embodiment, during the liquid chromatography-mass spectrometry analysis process, chromatographic column Cortecs HSS T3 100 mm is used in the liquid chromatography analysis process; the mobile phase used comprises phase A and phase B, wherein, the phase A is a mixed solution of water and formic acid, the phase B is a mixed solution of acetonitrile and formic acid, and the liquid chromatography elution program used in the liquid chromatography analysis process is a gradient elution program, the liquid chromatography elution program comprises a first equilibrium process, a first elution process, a second elution process, and a second equilibrium process sequentially; the volume of phase A is recorded as V_{A} and the volume of phase B is recorded as V_{B}, and the flow rate of the liquid chromatography mobile phase is recorded as Vₙ, with V₁ ranging from 0.2 to 0.6 mL/min. Preferably, in a mixed solution of water and formic acid, the volume ratio of water to formic acid is 200: (0.1-0.3); and preferably, in a mixed solution of acetonitrile and formic acid, the volume ratio of acetonitrile to formic acid is 200: (0.1-0.3). More preferably, phase A is a mixed solution of ultrapure water or filtered distilled water suitable for UPLC and chromatographically pure formic acid, while phase B is a mixed solution of chromatographically pure anhydrous acetonitrile and chromatographically pure formic acid. It should be additionally noted that the above conditions are the testing conditions for the Waters ACQUITY UPLC I-Class liquid chromatography analyzer, those skilled in the art can adjust and optimize the corresponding parameters based on practical experiences when using other liquid chromatography analyzers.

Considering the issue of residual derivating agents, for the purpose of instrument maintenance, in a preferred embodiment, the optional needle washing conditions are: 90% ACN+10% H₂O, once every 5-10 minutes or before/after each injection, for 5-10 seconds each time; and the optional column washing conditions are: after completing all sample tests, washing with H₂O for 5 minutes, CAN washing for 10-20 minutes, and a flow rate of 0.05 to 0.4 mL/min.

In one embodiment of the present disclosure, when testing small molecule carboxylic acid substances at any time point in the vital or metabolic activities of organisms in vivo, the liquid chromatography elution procedure is as follows: during the first equilibrium process, V_{A} is 70%-80%, V_{B} is 20%-30%, and the time of the first equilibrium process is 0-1 minute; during the first elution process, V_{A} is in a dynamic change process gradually switching from 70%-80% to 20%-30%, while V_{B} is in a dynamic change process gradually switching from 20%-30% to 70%-80%, and the time of the first elution process is 2-5 minutes; during the second elution process, V_{A} is in a dynamic change process gradually switching from 20%-30% to 0-5%, while V_{B} is in a dynamic change process gradually switching from 70%-80% to 95%-100%, and the time of the second elution process is 1-3 minutes; during the second equilibrium process, V_{A} is in a dynamic change process gradually switching from 0-5% to 70%-80%, while V_{B} is in a dynamic change process gradually switching from 95%-100% to 20%-30%, and the time of the second equilibrium process is 1-1.5 minutes. The test objects in the test sample include but are not limited to one or more of lactic acid, pyruvic acid, fumaric acid, oxaloacetic acid, α-ketoglutaric acid, succinic acid, malic acid, citric acid, or isocitric acid.

In another preferred embodiment, the liquid chromatography elution procedure is as follows: during the first equilibrium process, V_{A} is 45%-55%, V_{B} is 45%-55%, and the time of the first equilibrium process is 0-1 minute; during the first elution process, V_{A} is in a dynamic change process gradually switching from 45%-55% to 0-5%, while V_{B} is in a dynamic change process gradually switching from 45%-55% to 95%-100%, and the time of the first elution process is 3-4 minutes; during the second elution process, V_{A} is 0-5%, while V_{B} is 95%-100%, and the time of the second elution process is 2-4 minutes; during the second equilibrium process, V_{A} is in a dynamic change process gradually switching from 0-5% to 50%-55%, while V_{B} is in a dynamic change process gradually switching from 95%-100% to 45%-55%, and the time of the second equilibrium process is 1-1.5 minutes. The test objects in the test sample include fatty acids, which include but are not limited to one or more of myristic acid, palmitic acid, palmitoleic acid, stearic acid, oleic acid, linoleic acid, or arachidonic acid.

The present disclosure also provides use of the aforementioned diazo compound, or the diazo compound prepared by the aforementioned preparation method in drug screening related to the function of mitochondrial respiratory chain complexes.

The carboxylic acid metabolites in the tricarboxylic acid cycle refer to small molecule carboxylic acids with a molecular weight between 46 and 500; the small molecule carboxylic acids include one or more of myristic acid, palmitic acid, palmitoleic acid, stearic acid, oleic acid, linoleic acid, arachidonic acid, lactic acid, pyruvic acid, fumaric acid, oxaloacetic acid, α-ketoglutaric acid, succinic acid, malic acid, citric acid, or isocitric acid. In one embodiment of the present disclosure, the use includes in situ detection of metabolites in living cells using the diazo compound, and the detection includes the following steps: 1) inoculating 1×10⁴ primary neonatal rat cardiomyocytes in a 96-well cell plate, cell adherent, culturing for 1 day, then culturing in fresh culture medium for 2-3 days; discarding the culture medium of neonatal rat cardiomyocytes in the 96-well plate and washing the cells with substrate free culture medium more than two times; 2) using DMEM culture medium containing 20 mM of U—C13 glucose as substrate to label the primary neonatal rat cardiomyocytes for 0, 5 minutes, 10 minutes, 30 minutes, and 60 minutes, respectively; 3) discarding the DMEM culture medium containing 20 mM of U—C13 glucose mentioned above and adding 15-30 μL of a 1:1 (v/v) mixture of solution A and solution B aforementioned to each well of the 96-well plate to fully covering the surface of the primary neonatal rat cardiomyocytes, reacting at a temperature of 70° C. for 20 minutes, taking the supernatant of the reaction system and subjecting same to liquid chromatography-mass spectrometry analysis to calculate the ratio of M+2 and M+0, thereby obtaining the labeling information of the metabolites and throughput ratios flowing through different branching pathways.

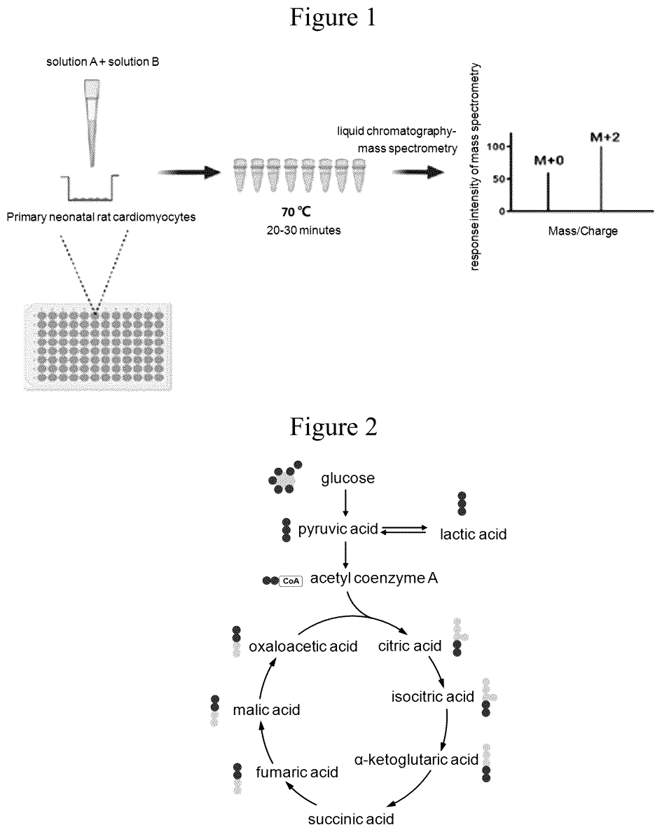

It should be additionally noted that the flowchart of the metabolic flow assay experiment of living cells in 96-well plate using U—C13-glucose as the carbon source is shown in FIG. 1, and the schematic diagram of its principle is shown in FIG. 2. Specifically, when U—C13 glucose is used as the carbon source to label cells, the newly generated acetyl coenzyme A is labeled with M+2, and the citric acid-isocitric acid and downstream metabolites generated by using acetyl coenzyme A as the entrance to enter the tricarboxylic acid cycle are labeled with M+2. The ratios of unlabeled form M+0 to labeled form M+2 of the key metabolites such as (iso)citric acid, pyruvic acid, fumaric acid, oxaloacetic acid, α-ketoglutaric acid, succinic acid, malic acid and lactic acid are calculated, respectively, so as to calculate the throughput ratios flowing through different branching pathways based on the labeling information of intermediate metabolites. Among them, when U—C13 glucose is not added, the abundance of C13 in all carboxylic acid metabolites is very low, almost all of the carboxylic acid metabolites are C12, which is labeled as M+0 at this point; after U—C13 glucose is added, glucose is decomposed and enters the tricarboxylic acid cycle, since it enters the tricarboxylic acid cycle in the form of pyruvic acid (containing two C13 atoms) each time, the C12 atom in the downstream carboxylic acid metabolites will gradually be replaced by C13, and the mass at this point will increase by 2, which is labeled as M+2. Therefore, based on the rate of change in the ratio of M+2 to M+0 over time, the rate changes at each step of the tricarboxylic acid cycle in cells (i.e. enzyme activity) can be inferred and then which step in the tricarboxylic acid cycle is inhibited or activated by a certain compound can be inferred.

By using the above diazo compound of the present disclosure as a derivatization reagent, the accurate determination of metabolic flow of unstable metabolites such as α-ketoglutaric acid and oxaloacetic acid, etc., in metabolites of living cells can be greatly improved, especially through derivatization reactions, which can fix the initial metabolic state of cells at the time point of response in situ through their chemical reactions, and make the responsiveness better, thus obtaining more stable and reliable metabolic flow data, and at the same time it has higher sensitivity in liquid chromatography-tandem mass spectrometry technology. Especially, based on the aforementioned derivatization reagents of the present disclosure, reliable results can be obtained in a small amount of cells, greatly reducing the cost of isotope labeling in metabolic flow experiments (glucose and fatty acids labeled with isotopes are relatively expensive). In addition, on the basis of the aforementioned diazo compound as a derivatization reagent, the present disclosure can effectively achieve in situ derivatization of cell samples, thereby shortening the treatment process of biological samples, reducing the degradation of small molecule carboxylic acid metabolites during the sample treatment process, and significantly improving the accuracy of detection results.

In one embodiment of the present disclosure, the above use includes screening drugs related to the function of mitochondrial respiratory chain complexes through in situ detection of living cell metabolism, wherein the drugs include an agonist and/or an inhibitor. Based on the in situ detection of living cell metabolism mentioned above, the influence of compounds on oxidative phosphorylation and glycolysis processes can be determined, which can be used for drug screening related to the function of mitochondrial respiratory chain complexes. Specifically, before using DMEM culture medium containing U—C13 glucose as substrate to culture cells, the drugs to be studied are added to treat primary neonatal rat cardiomyocytes. The promoting or inhibiting effects on oxidative phosphorylation and glycolysis by the drugs can be determined based on the isotopic labeling ratio of intracellular tricarboxylic acid cycle metabolites at different time points.

This method can quickly and high-throughput screen drugs targeting mitochondrial respiratory function, which is crucial for the screening of mitochondrial related drugs. Drugs that target mitochondria, whether promoting or inhibiting mitochondrial respiratory function, may become the drugs, depending on the specific pathogenesis of the diseases. For example, if the pathogenesis of a certain disease is caused by excessive enzyme activity of a certain enzyme during aerobic respiration, inhibitors should be sought; while if the pathogenesis is a decrease in enzyme activity during aerobic respiration, activators should be sought. The present disclosure provides a high-throughput screening scheme for searching for potential mitochondrial respiratory agonists or inhibitors, which is very beneficial for subsequent drug development.

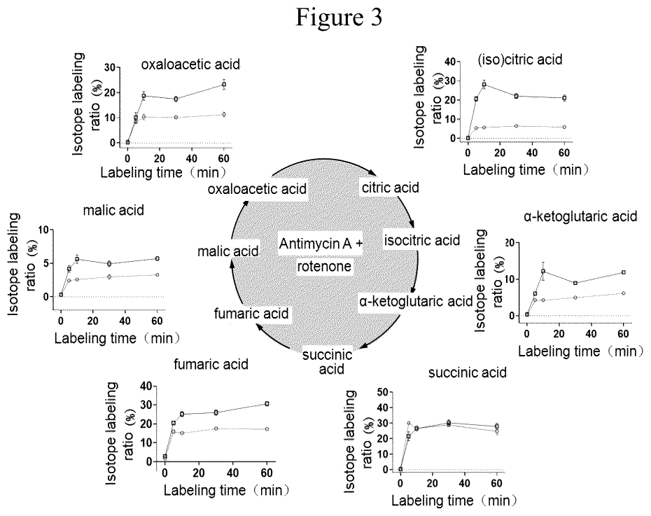

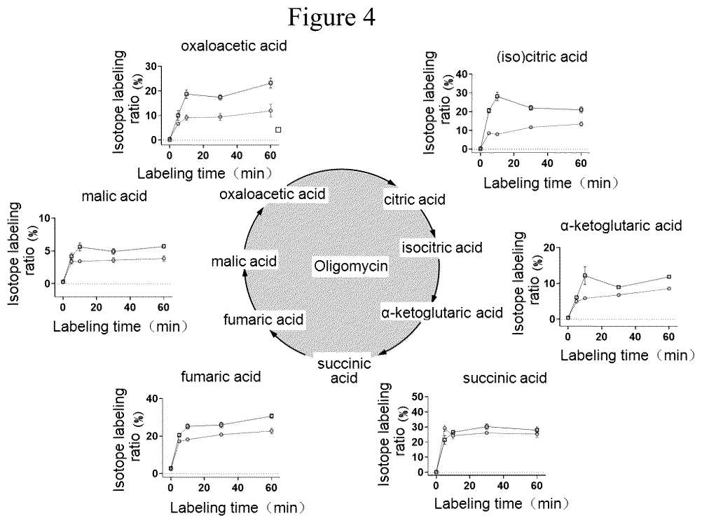

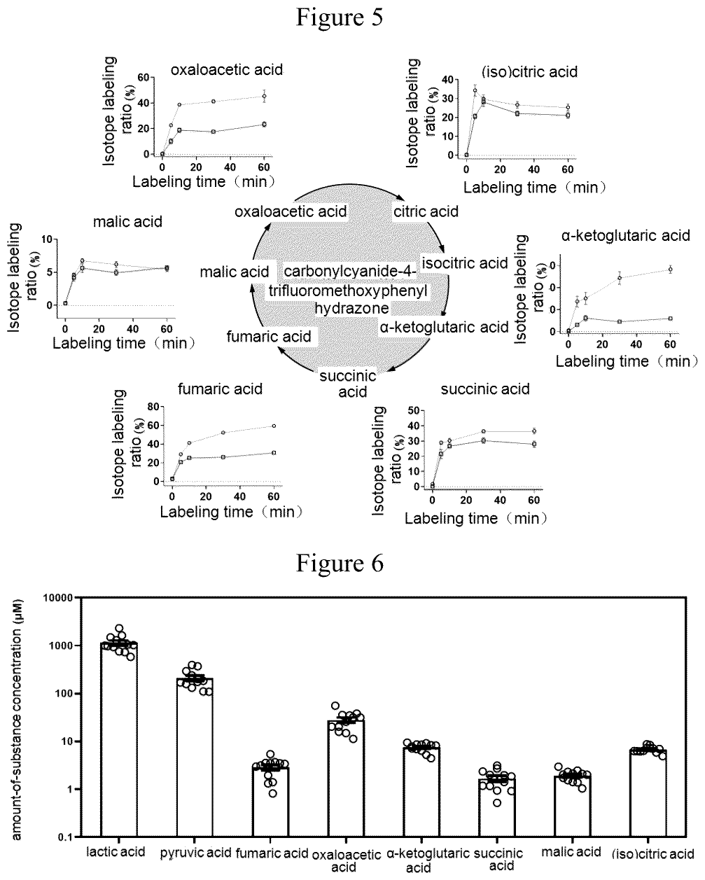

For example, in some embodiments of the present disclosure, when both the inhibitor of mitochondrial respiratory chain complex I (Rotenone), and the inhibitor of complex III (Antimycin A) are used to simultaneously treat neonatal rat cardiomyocytes, both of which significantly inhibit the metabolic flow of other tricarboxylic acid cycle substrates except for succinic acid, as shown in FIG. 3. In one embodiment of the present disclosure, when the inhibitor (oligomycin) of mitochondrial respiratory chain complex V (ATPase) is used to treat neonatal rat cardiomyocytes, the metabolic flows of oxaloacetic acid and isocitric acid are significantly inhibited, and the metabolic flows of α-ketoglutaric acid, fumaric acid, and malic acid are inhibited to some extent, as shown in FIG. 4. In one embodiment of the present disclosure, the uncoupling agent carbonylcyanide-4-trifluoromethoxyphenylhydrazone (FCCP) is used to treat neonatal rat cardiomyocytes, which significantly enhances the metabolic flows of oxaloacetic acid, α-ketoglutaric acid and fumaric acid, as shown in FIG. 5.

It should be noted that the correlation between derivatization treatment and labeling rate (or the rate of change in labeling ratio) is as follows: in situ derivatization can directly fix the metabolites at the time of reagent addition, without the need for step-by-step cell metabolism termination or cell collection and extraction processes, which can theoretically minimize the degradation of unstable metabolites, making the determination of labeling rate more reliable. The significant improvement in sensitivity requires only a small amount of cells (such as 96-well plates) to be cultured in the embodiment, which reduces the usage amount of cells and at the same time proportionally reduces the addition of expensive isotope labeled nutrients (such as U—C13 glucose) and drugs to be studied during the cell culture process, greatly reducing study costs.

In one embodiment of the present disclosure, 0.5-2 mL of normal human blood is collected by venous blood sampling and placed on ice; 2) centrifuged at a speed of 1,500-4,500 rpm and a temperature of 4-25° C. for 10-30 minutes; 3) the supernatant after centrifugation is collected as the plasma to be tested, and stored at −20° C. to −80° C.; 4) formulating a solution of 20-25 mM of derivatization reagent in acetonitrile and labeled as solution A, and formulating an aqueous solution of 20-25 mM of hydroxylamine hydrochloride and labeled as solution B; 5) 20-32 μL of solution A is added to 5-8 μL of the plasma to be tested (4-fold of the volume of plasma to be tested), which are mixed well and centrifuged, 20 μL of supernatant is aspirated and 20-60 μL of solution B is added, which are mixed well and heated at 70° C. for 20 minutes, after centrifugation, the supernatant is aspirated for the above liquid chromatography-mass spectrometry analysis. The absolute content of tricarboxylic acid cycle metabolites in normal human serum measured using the above method is shown in FIG. 6. The summary of the absolute content data of tricarboxylic acid cycle metabolites in normal human serum in the HMDB (Human Metabolite Database) public database is shown in the table below.

| Tricarboxylic acid | Normal concentration | |
|---|---|---|
| cycle metabolites | range in adult blood | |
| L-lactic acid | 740-2400 | μM |
| Pyruvic acid | 22-258 | μM |
| Oxaloacetic acid | No data | |
| α-ketoglutaric acid | 0.0-23.0 | μM |
| Succinic acid | 0.0-32.0 | μM |
| Fumaric acid | 0.0-4.0 | μM |
| Malic acid | 0.0-21.0 | μM |
| Citric acid | 30.0-400.0 | μM |
| Isocitric acid | 0.0-10.0 | μM |

By comparing FIG. 6 with the summary of absolute content data in the above table, it can be found that the above carboxylic acid content measured by the above detection method of the present disclosure is reasonable and accurate. Among them, although there is no data on the content of oxaloacetic acid in the public database, the concentration of micromoles per liter is relatively reasonable based on the order of magnitude of the measured data.

Generally, mitochondria are the main site for the tricarboxylic acid cycle, and mitochondrial diseases are diseases with abnormal mitochondrial function in multiple organs or tissues within the human body. Therefore, we believe that the tricarboxylic acid cycle metabolites in plasma can reflect mitochondrial function in the human body. On this basis, those skilled in the art can use the tricarboxylic acid cycle metabolites in human serum measured by the above method to reflect the mitochondrial function in the human body.

The present disclosure also provides a kit, which includes the aforementioned diazo compounds or diazo compounds prepared by the aforementioned preparation method.

In one embodiment of the present disclosure, the operation instruction for the above kit is as follows: 3-5 mL of test subject blood is collected by venous blood sampling and placed on ice; 2) centrifuged at a speed of 1,500-4,500 rpm and a temperature of 4-25° C. for 10-30 minutes; 3) the supernatant after centrifugation is collected as the plasma to be tested, and stored at −80° C.; 4) formulating a solution of 20-100 mM of derivatization reagent (i.e. the above diazo compounds) in acetonitrile and labeled as solution A, and formulating an aqueous solution of 20-25 mM of hydroxylamine hydrochloride and labeled as solution B; 5) 20-32 μL of solution A is added to 5-8 μL of the plasma to be tested, which are mixed well and centrifuged, 20 μL of supernatant is aspirated and 20-60 μL of solution B is added, which are mixed well and heated at 70° C. for 20 minutes, after centrifugation, the supernatant is aspirated for the above liquid chromatography-mass spectrometry analysis. On this basis, the present disclosure can perform rapid initial screening of test subjects, and provide a more stable, reliable, fast, convenient, and low-cost method and approach for subsequent clinical sample detection, clinical disease diagnosis, efficacy evaluation, and prognosis evaluation.

The disclosure will be further described in detail below in combination with specific examples, which cannot be understood as limiting the scope of protection claimed in the application.

### Example 1

### Preparation of DQmB

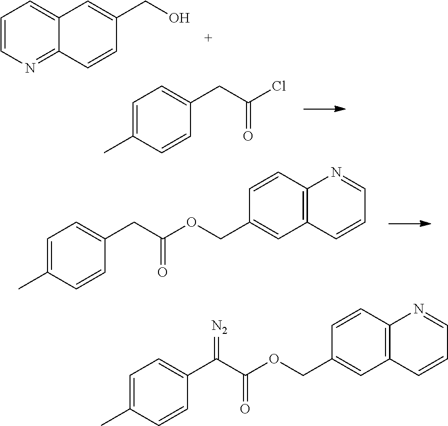

Quinolinol (6 g, 1 equiv.) was dissolved in dichloromethane (36 mL), triethylamine (13.1 mL, 2.5 equiv.) was added and p-methylphenylacetyl chloride (6.99 g, 1.1 equiv.) was slowly added at low temperature. The reaction mixture was stirred at 5-10° C. for 8-12 hours. The reaction solution was poured into pure water (30 mL), the aqueous phase was separated, which was extracted twice sequentially with dichloromethane (15 mL and 10 mL). The organic phases were combined, dried over anhydrous sodium sulfate, filtered, and rotary evaporated to dryness under reduced pressure. The dried substance obtained from rotary evaporation was purified by column chromatography to obtain quinoline p-methylphenylacetate (6.8 g). The aforementioned quinoline p-methylphenylacetate was dissolved in acetonitrile (45 mL), to which p-ABSA (9.21 g, 2 equiv.) and DBU (10.7 g, 3 equiv.) were added sequentially at low temperature. The reaction mixture was stirred at 4-8° C. for 8-10 hours. The reaction solution was concentrated under reduced pressure, and rotary evaporated to dryness under reduced pressure, the crude product was purified by column chromatography to obtain the target compound DQmB (5.8 g, purity of 95.1%). The nuclear magnetic resonance data of the product:

¹HNMR (400 MHz, CDCl₃): δ 8.94 (dd, J=1.6, 4.4 Hz, 1H), 8.16-8.12 (m, 2H), 7.84 (d, J=0.8 Hz, 1H), 7.75 (dd, J=2.0, 4.0 Hz, 1H), 7.44-7.37 (m, 3H), 7.21 (d, J=8.0 Hz, 2H), 5.49 (s, 2H), 2.35 (s, 3H). ¹³CNMR (101 MHz, CDCl₃): δ 165.1, 150.8, 148.0, 136.1, 135.9, 134.2, 130.0, 129.7, 129.2, 128.0, 127.0, 124.2, 121.8, 121.4, 66.0, 21.0.

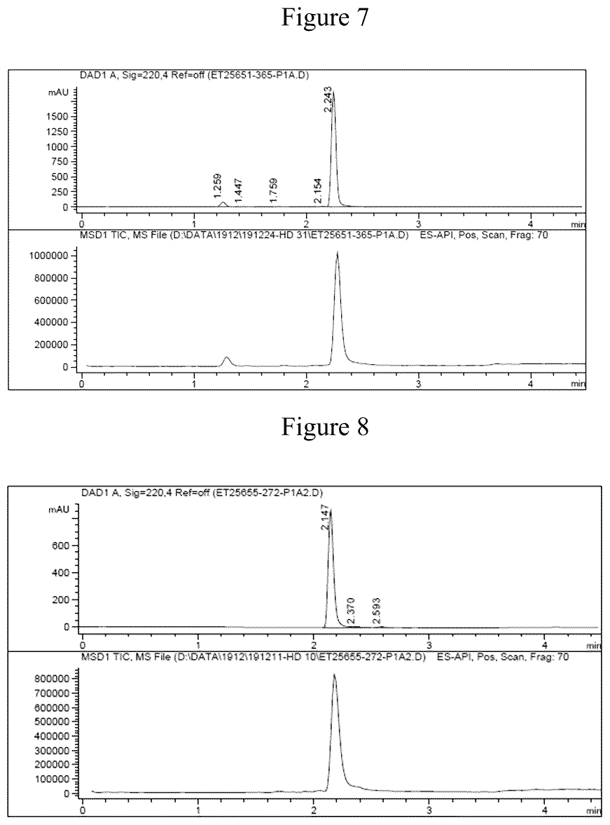

LC/MS is as shown in FIG. 7.

### Example 2

### Preparation of DDAB

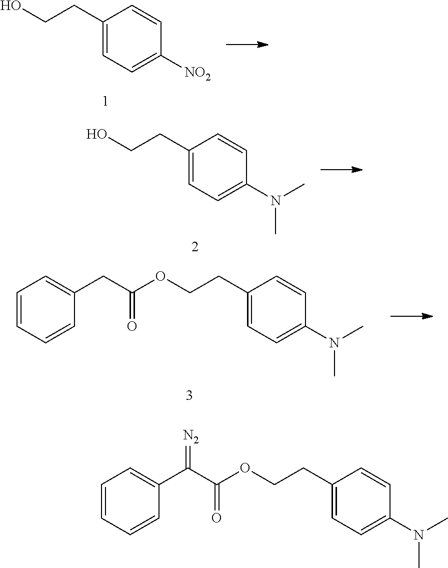

The p-(N,N-dimethylamino) aminoethyl benzyl alcohol was dissolved in dichloromethane, and triethylamine (2 equiv.) and phenylacetyl chloride (1.1 equiv.) were added sequentially at low temperature. The reaction mixture was stirred at 5-10° C. for 8-12 hours. The reaction solution is poured into pure water, the aqueous phase is separated, which is extracted twice sequentially with dichloromethane. The organic phases were combined, dried over anhydrous sodium sulfate, filtered, and rotary evaporated to dryness under reduced pressure. The dried substance obtained from rotary evaporation was purified by column chromatography to obtain (N,N-dimethylamino)phenethyl phenylacetate. The aforementioned phenylacetate was dissolved in acetonitrile, and TsN₃ (2 equiv.) and DBU (3 equiv.) were added sequentially at low temperature. The reaction mixture was stirred at 4-8° C. for 8-10 hours. The reaction solution was concentrated under reduced pressure, and rotary evaporated to dryness under reduced pressure, the crude product was purified by column chromatography to obtain the target compound DDAB. The nuclear magnetic resonance data of the product:

¹HNMR (400 MHz, CDCl₃): δ 7.48-7.46 (d, J=7.6 Hz, 2H), 7.40-7.37 (m, 2H), 7.21-7.19 (m, 1H), 7.13 (d, J=8.4 Hz, 2H), 6.73 (d, J=8.4 Hz, 2H), 4.43 (t, J=7.2 Hz, 2H), 2.95-2.92 (m, 8H). ¹³CNMR (101 MHz, CDCl₃): δ 165.1, 149.5, 129.6, 128.9, 125.7, 125.6, 125.4, 124.0, 112.9, 66.0, 40.7, 34.3.

LC/MS is as shown in FIG. 8.

Liquid chromatography analysis can be carried out according to the following procedures:

A solution of 20 mM of DQmB in acetonitrile (solution A) and aqueous solution of 20 mM of hydroxylamine hydrochloride (solution B) were formulated.

5 μL of mouse plasma was taken and placed into a 200 μL of centrifuge tube, 4-fold volume of solution A was added, shaken and mixed well, the mixture was centrifuged at 12,000 rpm, 4° C. for 5 minutes, the supernatant was taken and placed into a 200 μL of centrifuge tube, 2-fold volume of solution B was added, and heated at 70° C. for 20 minutes, the mixture was centrifuged at 12,000 rpm, 4° C. for 5 minutes before loading.

During the liquid chromatography analysis process: the chromatographic column used was CortecsHSST3100 mm; the mobile phase used included phases A and B, wherein phase A was 200 mL of ultrapure water plus 0.1% (v/v) formic acid; and phase B was 200 mL of acetonitrile plus 0.1% (v/v) formic acid. Chromatographic column: CortecsHSST3100 mm.

The test objects in the test sample included but were not limited to one or more of lactic acid, pyruvic acid, fumaric acid, oxaloacetic acid, α-ketoglutaric acid, succinic acid, malic acid, citric acid, or isocitric acid, and the elution conditions are shown in the table below:

| | Flow | | | Gradient |
|---|---|---|---|---|
| Time/min | rate/mL · min⁻¹ | A/% | B/% | change curve |
| 0 | 0.4 | 75 | 25 | — |
| 0.5 | 0.4 | 75 | 25 | 11 |
| 5 | 0.4 | 25 | 75 | 7 |
| 7 | 0.4 | 0 | 100 | 1 |
| 8 | 0.4 | 75 | 25 | 1 |

Note: The gradient change curve is a built-in gradient change curve in Waters liquid chromatography-mass spectrometer, which represents the change rate of phase B. There are a total of 11 curves, labeled with Nos. 1-11, wherein No. 6 is a straight line, that is, the ratio of phase B changes uniformly over time; the curves of Nos. 1-5 indicate that the change is fast first and then slow, with the curve of No. 1 almost instantly changing to the target ratio; the curves of Nos. 7-1 indicate that the change is slow first and then fast, with the curve of No. 11 showing extremely slow changes at the beginning and extremely fast changes in the later stages.

During the mass-spectrometry analysis process, a triple quadrupole mass spectrometer was used.

The parameters for mass-spectrometry are shown in the table below:

| Parameters | Numerical values |
|---|---|
| Source temperature (° C.) | 150 |
| Cone voltage (kV) | 30 |
| Cone gas flow rate (L/Hr) | 20 |
| Capillary voltage (kV) | 2.00 |
| Desolvation temperature (° C.) | 400 |
| Desolvation gas flow rate (L/Hr) | 1000 |

The information on the determination of parent ions, daughter ions, and collision energy by mass-spectrometry is shown in the table below.

| Name of | Parent | Daughter | Residence | Cone | Collision |
|---|---|---|---|---|---|
| compounds | ions | ions | time | voltage | energy |
| Lactic acid | 380.17 | 142.14 | 0.02 | 28 | 28 |
| Pyruvic acid | 393.3 | 142.1 | 0.02 | 22 | 28 |
| Fumaric acid | 406.16 | 142.17 | 0.02 | 40 | 28 |
| Oxaloacetic acid | 437.11 | 142.22 | 0.02 | 28 | 46 |
| α-ketoglutaric | 451.3 | 142.09 | 0.02 | 40 | 40 |
| acid | | | | | |
| Succinic acid | 408.17 | 142.07 | 0.02 | 28 | 34 |
| Malic acid | 424.16 | 142.07 | 0.02 | 34 | 34 |
| (Iso)citric acid | 482.29 | 142.19 | 0.02 | 34 | 34 |
| α-Ketovaline | 421.21 | 232.13 | 0.02 | 40 | 16 |
| α-Keto | 435.21 | 142.13 | 0.02 | 40 | 40 |
| (iso)leucine | | | | | |
| α-Hydroxybutyric | 394.15 | 158 | 0.02 | 22 | 40 |
| acid | | | | | |
| β-Hydroxybutyric | 394.11 | 141.9 | 0.02 | 40 | 22 |
| acid | | | | | |
| Acetoacetic acid | 407.3 | 141.7 | 0.02 | 20 | 40 |

The test objects in the test sample include but are not limited to one or more of myristic acid, palmitic acid, palmitoleic acid, stearic acid, oleic acid, linoleic acid, or arachidonic acid, and the elution conditions are shown in the table below:

| | Flow | | | |
|---|---|---|---|---|
| Time/min | rate/mL · min⁻¹ | A/% | B/% | Curve |
| 0 | 0.4 | 50 | 50 | — |
| 0.5 | 0.4 | 50 | 50 | 11 |
| 4 | 0.4 | 0 | 100 | 6 |
| 7 | 0.4 | 0 | 100 | 1 |
| 8 | 0.4 | 50 | 50 | 1 |

The information on the determination of parent ions, daughter ions, and collision energy by mass-spectrometry is shown in the table below.

| Name of | Parent | Daughter | Residence | Cone | Collision |
|---|---|---|---|---|---|
| compounds | ions | ions | time | voltage | energy |
| Myristic acid | 518.4824 | 142 | 0.021 | 40 | 40 |
| Palmitic acid | 546.4546 | 142 | 0.021 | 50 | 46 |
| Palmitoleic | 544.5642 | 141.8262 | 0.021 | 34 | 34 |
| acid | | | | | |
| Stearic acid | 574.5212 | 142 | 0.021 | 40 | 30 |
| Oleic acid | 572.5526 | 142.1425 | 0.021 | 34 | 40 |
| Linoleic acid | 570.6472 | 142.3305 | 0.021 | 50 | 40 |
| Arachidonic | 594.555 | 142.0869 | 0.021 | 28 | 46 |
| acid | | | | | |

Characterization of sensitivity:

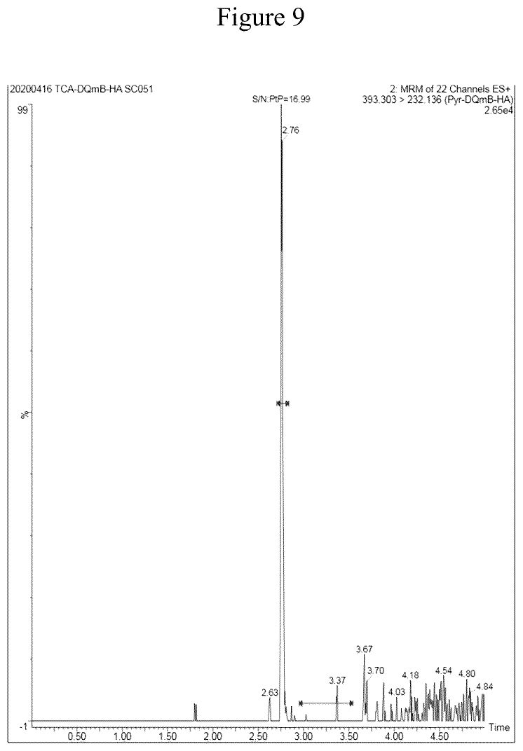

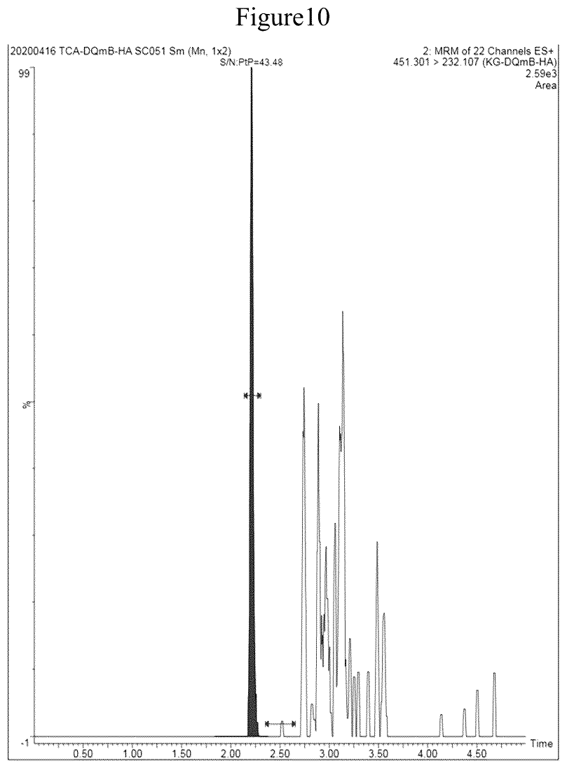

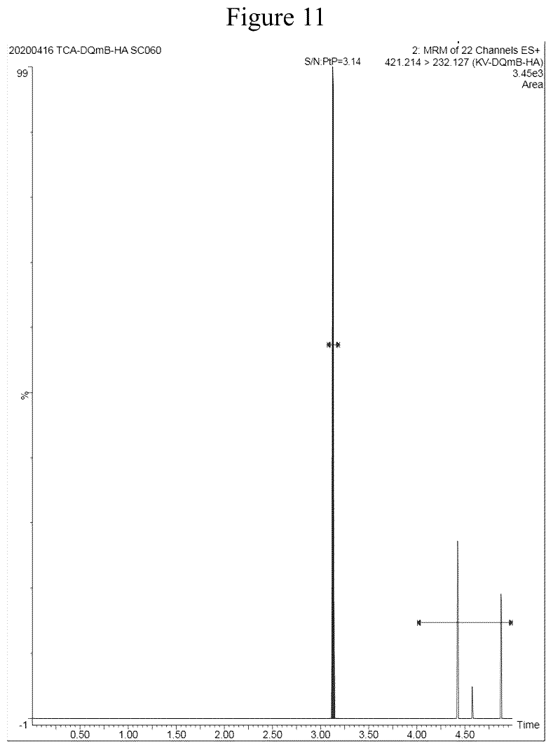

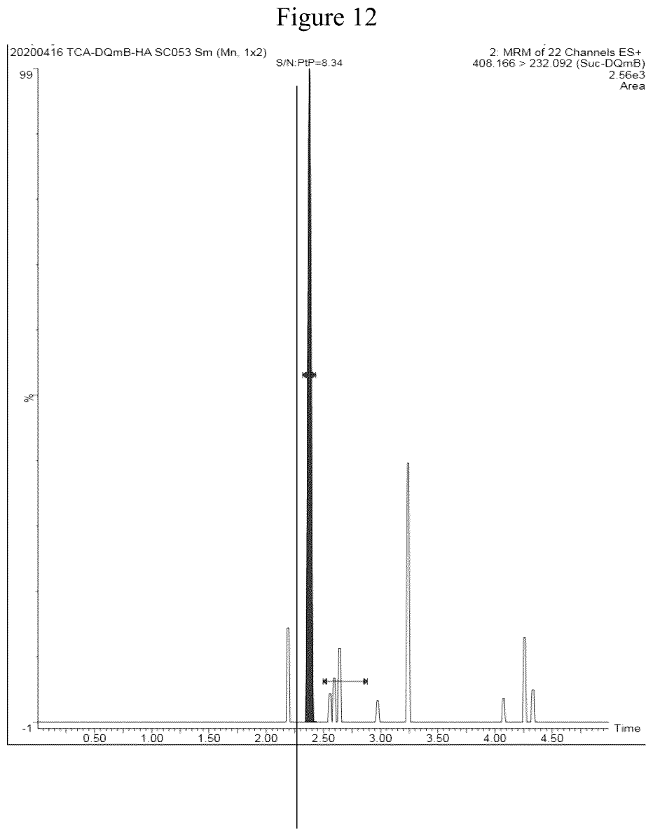

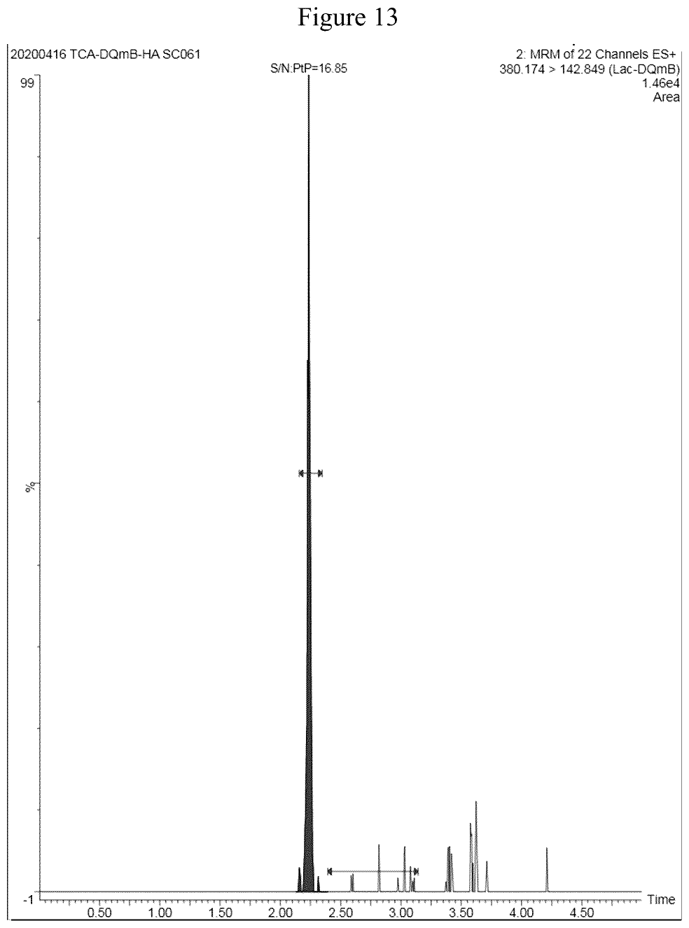

The acid concentration at signal-to-noise ratio (S/N=3.3) of the mass-spectrometry is the detection limit. Among them, FIG. 9 shows the secondary mass spectrogram-chromatogram of the pyruvic acid standard sample (1 nM) derivatized with the diazo compound in example 1 of the present disclosure; FIG. 10 shows the secondary mass spectrogram-chromatogram of the α-ketoglutaric acid standard sample (1 nM) derivatized with the diazo compound in example 1 of the present disclosure; FIG. 11 shows the secondary mass spectrogram-chromatogram of the α-ketovaline standard sample (2 nM) derivatized with the diazo compound in example 1 of the present disclosure; FIG. 12 shows the secondary mass spectrogram-chromatogram of the succinic acid standard sample (5 nM) derivatized with the diazo compound in example 1 of the present disclosure; and FIG. 13 shows the secondary mass spectrogram-chromatogram of the lactic acid standard sample (5 nM) derivatized with the diazo compound in example 1 of the present disclosure. From FIGS. 9 to 13, it can be found that the signal-to-noise ratios of the secondary mass-spectrometry are 17, 43, 3, 8, and 17, respectively. This means that the detection limits of each acid are not higher than the labeled concentrations in the figure (which are 1 nM, 1 nM, 2 nM, 5 nM, and 5 nM, respectively).

1-20. (canceled)

21. A diazo compound, wherein, the diazo compound has the structure as shown in formula I: 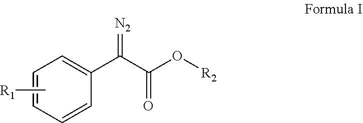
in formula, R₁ represents H, alkyl, halogen, alkoxy or alkylamino; and R₂ represents an aromatic group.

22. The diazo compound according to claim 21, wherein, R₁ represents H, C₁-C₆ alkyl, halogen, C₁-C₆ alkoxy or dimethylamino; and R₂ represents methylenequinolyl or ethyl-N,N-dimethylanilino group.

23. A preparation method of the diazo compound according to claim 21, wherein, the preparation method comprises:
performing an esterification reaction on a first dispersion containing a phenylacetic acid compound and an alcohol compound to generate an intermediate product A;
performing a diazotization reaction on a second dispersion containing the intermediate product A and an diazo transfer reagent to generate a diazo compound;
wherein, the phenylacetic acid compound has the structure as shown in 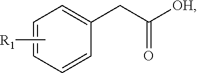
and the alcohol compound has the structure as shown in R₂—OH; R₁ represents H, alkyl, halogen, alkoxy or alkylamino; and R₂ represents an aromatic group.

24. The preparation method according to claim 23, wherein, during the esterification reaction process, the reaction temperature is from 0° C. to 30° C. and the reaction time is from 0.5 hours to 24 hours.

25. A detection method for quantitative analysis of a small molecule carboxylic acid, wherein the small molecule carboxylic acid represents a carboxylic acid with a molecular weight between 46 and 500, wherein, the detection method comprises:
derivatization treatment involving derivatizing a sample containing the small molecule carboxylic acid with a derivatization reagent to obtain a derivatized sample;
liquid chromatography-mass spectrometry analysis involving performing liquid chromatography-mass spectrometry analysis on the derivatized sample to obtain a liquid chromatogram-mass chromatogram, and quantitatively analyzing the small molecule carboxylic acid components in the sample based on the liquid chromatogram-mass chromatogram; wherein, the derivatization reagent is the diazo compound of claim 21.

26. The detection method for quantitative analysis of a small molecule carboxylic acid according to claim 25, wherein, the derivatization treatment comprises:
formulating 20-100 mM of an acetonitrile solution of the derivatization reagent and recording it as solution A;
formulating 20-100 mM of an aqueous solution of the hydroxylamine compound and recording it as solution B;
mixing the sample containing the small molecule carboxylic acid with the solution A, centrifuging the mixture at 10,000 to 17,000 rpm and a temperature of 4° C. to 30° C. for 5 minutes to 8 minutes, then mixing the supernatant after centrifugation with the solution B, and subjecting the sample to derivatization reaction at a temperature of 50° C. to 80° C. to obtain the derivatized sample; preferably the time for the derivatization reaction being 10 minutes to 60 minutes.

27. The detection method for quantitative analysis of a small molecule carboxylic acid according to claim 25, wherein, during the liquid chromatography-mass spectrometry analysis process, the mobile phase used for the liquid chromatography analysis comprises phase A and phase B, wherein, the phase A is a mixed solution of water and formic acid, the phase B is a mixed solution of acetonitrile and formic acid, and the liquid chromatography elution program used in the liquid chromatography analysis process is a gradient elution program, the liquid chromatography elution program comprises a first equilibrium process, a first elution process, a second elution process, and a second equilibrium process sequentially; recording the volume of phase A as V_{A} and the volume of phase B as V_{B}, and recording the flow rate of the liquid chromatography mobile phase as Vₙ, with V₁ ranging from 0.2 to 0.6 mL/min.

28. The detection method for quantitative analysis of a small molecule carboxylic acid according to claim 27, wherein, during the first equilibrium process, V_{A} is 70%-80%, V_{B} is 20%-30%, and the time of the first equilibrium process is 0-1 minute; during the first elution process, V_{A} is in a dynamic change process gradually switching from 70%-80% to 20%-30%, while V_{B} is in a dynamic change process gradually switching from 20%-30% to 70%-80%, and the time of the first elution process is 2-5 minutes; during the second elution process, V_{A} is in a dynamic change process gradually switching from 20%-30% to 0-5%, while V_{B} is in a dynamic change process gradually switching from 70%-80% to 95%-100%, and the time of the second elution process is 1-3 minutes; during the second equilibrium process, V_{A} is in a dynamic change process gradually switching from 0-5% to 70%-80%, while V_{B} is in a dynamic change process gradually switching from 95%-100% to 20%-30%, and the time of the second equilibrium process is 1-1.5 minutes; or during the first equilibrium process, V_{A} is 45%-55%, V_{B} is 45%-55%, and the time of the first equilibrium process is 0-1 minute; during the first elution process, V_{A} is in a dynamic change process gradually switching from 45%-55% to 0-5%, while V_{B} is in a dynamic change process gradually switching from 45%-55% to 95%-100%, and the time of the first elution process is 3-4 minutes; during the second elution process, V_{A} is 0-5%, while V_{B} is 95%-100%, and the time of the second elution process is 2-4 minutes; during the second equilibrium process, V_{A} is in a dynamic change process gradually switching from 0-5% to 50%-55%, while V_{B} is in a dynamic change process gradually switching from 95%-100% to 45%-55%, and the time of the second equilibrium process is 1-1.5 minutes.

29. Use of the diazo compound according to claim 21 in drug screening related to the function of mitochondrial respiratory chain complexes.

30. The use according to claim 29, wherein, the diazo compound is used for in situ detection of metabolites of living cells, and screening drugs related to the function of mitochondrial respiratory chain complexes.

31. A kit, wherein the kit comprises the diazo compound according to claim 21.

32. The preparation method according to claim 23, wherein, during the diazotization reaction process, the reaction temperature is from 0° C. to 30° C. and the reaction time is from 1 hour to 24 hours;
and/or, during the esterification reaction process, the molar ratio of the phenylacetic acid compound to the alcohol compound is (0.5-2): 1;
and/or, during the diazotization reaction process, the molar ratio of the intermediate product A to the diazo transfer reagent is (1-3): 1;
and/or, during the diazotization reaction process, the diazo transfer reagent is one or more of 4-acetamidobenzenesulfonyl azide, p-toluenesulfonyl azide, 4-carboxybenzenesulfonyl azide, 1H-imidazole-1-sulfonyl azide hydrochloride, or 2-azido-1,3-dimethylimidazolium hexafluorophosphate; and/or, the first dispersion comprises a first solvent, which is one or more of dichloromethane, trichloromethane, N, N-dimethylformamide, tetrahydrofuran, or diethyl ether; and/or, the first dispersion also comprises a first catalyst, which is one or more of triethylamine, N,N-diisopropylethylamine, or alkali carbonate;
and/or, the second dispersion comprises a second solvent, which is acetonitrile and/or dimethyl sulfoxide.

33. The preparation method according to claim 23, wherein, the second dispersion also comprises a second catalyst.

34. The preparation method according to claim 33, wherein, the second catalyst is one or more of 1,8-diazabicyclo[5.4.0]undec-7-ene, triethylamine, sodium bicarbonate, sodium carbonate, potassium carbonate, potassium hydroxide or potassium acetate.

35. The preparation method according to claim 26, wherein, the sample is plasma, serum, urine, tears, tissue fluid, cells, tissue homogenate, bacterial culture medium, blood plaque or feces; and/or, the small molecule carboxylic acid is one or more of myristic acid, palmitic acid, palmitoleic acid, stearic acid, oleic acid, linoleic acid, arachidonic acid, lactic acid, pyruvic acid, fumaric acid, oxaloacetic acid, α-ketoglutaric acid, succinic acid, malic acid, citric acid, or isocitric acid.

36. The preparation method according to claim 27, wherein, in the mixed solution of water and formic acid, the volume ratio of water to formic acid is 200: (0.1-0.3); and/or, in the mixed solution of acetonitrile and formic acid, the volume ratio of acetonitrile to formic acid is 200: (0.1-0.3).

37. The preparation method according to claim 30, wherein, the drug comprises an agonist and/or an inhibitor; and/or, the metabolites are carboxylic acid metabolites in the tricarboxylic acid cycle.

38. The preparation method according to claim 37, wherein, the carboxylic acid metabolites in the tricarboxylic acid cycle refer to a small molecule carboxylic acid with a molecular weight between 46 and 500.

39. The preparation method according to claim 38, wherein, the small molecule carboxylic acid is one or more of myristic acid, palmitic acid, palmitoleic acid, stearic acid, oleic acid, linoleic acid, arachidonic acid, lactic acid, pyruvic acid, fumaric acid, oxaloacetic acid, α-ketoglutaric acid, succinic acid, malic acid, citric acid, or isocitric acid.

40. The preparation method according to claim 39, wherein, the detection method for quantitative analysis of a small molecule carboxylic acid according to claim 5 is used for in situ detection.
